(19) United States
(12) Patent Application Publication
(10) Pub. No.: US 2026/0250225 A1
(43) Pub. Date: Aug. 27, 2026

(54) PROCESS FOR THE PREPARATION OF ETHYLHEXYLGLYCEROL

(71) Applicant: SYMRISE AG, Holzminden (DE)

(72) Inventors: Dietmar SCHATKOWSKI, Einbeck (DE); Stefan BRAND, Vahlbruch (DE); Nikolas BUGDAHN, Holzminden (DE); Artur DÜCK, Stadtoldendorf (DE); Nicklas RATAY, Holzminden (DE)

(73) Assignee: SYMRISE AG, Holzminden (DE)

(21) Appl. No.: 19/160,105

(22) PCT Filed: Mar. 6, 2023
(86) PCT No.: PCT/EP2023/055561

§ 371 Date: Aug. 27, 2025

(51) Int. Cl. C07C 43/13; C07C 41/26; C07C 41/42

(52) U.S. Cl. CPC C07C 43/13; C07C 41/26; C07C 41/42

(57) ABSTRACT

The present invention primarily relates to a process for the preparation of ethylhexylglycerol of formula (I) as defined herein and to ethylhexylglycerol of formula (I) as defined herein obtained or obtainable according to a process as defined herein.

The present invention primarily relates to a process for the preparation of ethylhexylglycerol of formula (I) as defined herein and to ethylhexylglycerol of formula (I) as defined herein obtained or obtainable according to a process as defined herein.

Further aspects of the present invention will arise from the description below, in particular from the examples, as well as from the attached patent claims.

Ethylhexylglycerol (IUPAC: 3-[(2-Ethylhexyl)oxy]-1,2-propanediol; CAS: 70445-33-9; in the literature often also called ethylhexylglycerin) is a multifunctional substance for use in cosmetic, pharmaceutical, and household products and is mainly used as deodorant active ingredient. It inhibits the growth of odour-causing bacteria while being gentle to the skin at the same time. Moreover, it can boost the efficacy of traditional preservatives and acts as an emollient and mild humectant. Given to its multifunctional benefits, such as bacteriostatic and moisturising effects, various synergistic effects of ethylhexylglycerol can be used in formulations, including emulsions, wipes and gels, as well as in surfactant-based products, such as shampoos and shower gels.

Several publications are known from the state of the art dealing with various types of syntheses for the preparation of ethylhexylglycerol. Most major industrial approaches start from the same starting material: 2-Ethylhexyl glycidyl ether (IUPAC: 2-(2-ethylhexoxymethyl) oxirane; CAS: 2461-15-6).

A process described in KR 101878433 B1 performs the reaction of 2-ethylhexyl glycidyl ether to ethylhexylglycerol in water in a hydrolysis step. The reaction proceeds at 120° C. with the addition of small amounts of formic acid, sodium hydroxide and an additional organic solvent within 12 hours. While on the one hand the reduced use of formic acid and sodium hydroxide lowers the costs for these compounds, on the other hand the process costs increase due to the need for solvents (disadvantageous space-time yield). A similar process is shown in CN 104402682 A, where the reaction starts with the addition of iso-octyl alcohol and epichlorohydrin to give the 2-ethylhexyl glycidyl ether. Then, hydrolysis takes place in water with the addition of sodium acetate at 90° C. for 10 hours. WO 2018/194429 A1 uses higher temperatures (220° C.) as the driving force to enable the hydrolysis step with a high excess of water and without any further additives. The water used as a solvent plays a crucial role in the success of said three described processes, as more dimerization and product loss occur in the absence of solvent. The dilute reaction systems combined with the long reaction times, however, make the processes impractical for use on an industrial scale.

DE 102009032235 A1 and EP 2277849 B1 describe a synthesis of ethylhexylglycerol starting from 2-ethylhexyl glycidyl ether in a one-pot synthesis for the epoxide ring opening step and the transesterification step with subsequent distillation. In said publications, the importance of the presence of a strong acid, such as sulfuric acid, and of an excess of a carbonic acid (preferably formic acid) in the acylation step as well as the access to an alcohol (preferably methanol) in the following transesterification step are highlighted. Dry conditions as well as moderate temperatures lead to the product, which is obtained after alkalizing agent is used for neutralisation prior to distillation. This process has numerous disadvantages, such as
the use of strong, corrosive acids (such as sulfuric acid)
the creation of large amounts of formic acid waste due to its use in excess
an extra transesterification step with long distillation time and the use of toxic methanol
a possible lack of neutral odour of the obtained ethylhexylglycerol.

It was therefore an object of the present invention to provide a process for the preparation of ethylhexylglycerol that would further improve the odour profile of ethylhexylglycerol, overcome the economic disadvantages described above, and improve the efficiency of the synthesis of ethylhexylglycerol.

Further objects underlying the present invention follow from the description below and the present patent claims.

According to a first aspect of the present invention, the stated objects are achieved by a process for the preparation of ethylhexylglycerol of formula (I)

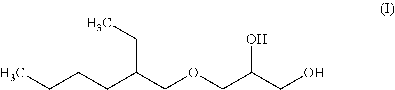

comprising or consisting of the following steps:
(a) Providing 2-ethylhexyl glycidyl ether of formula (II)

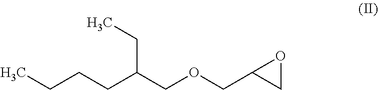

and formic acid,
(b) adding, preferably dropwise or portionwise, the 2-ethylhexyl glycidyl ether of formula (II) provided in step (a) to the formic acid provided in step (a) to give one or more compound(s) selected from the group consisting of compound (III), compound (IV), and compound (V)

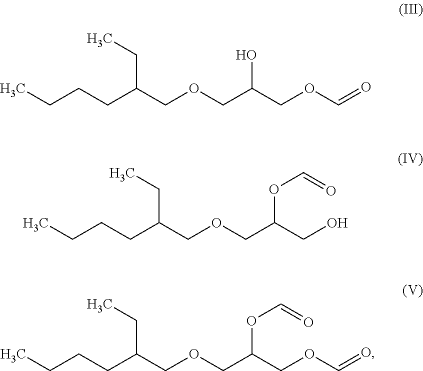 [SMILES: CCCCC(CC)COCC(O)COC=O ; CCCCC(CC)COCC(CO)OC=O ; CCCCC(CC)COCC(COC=O)OC=O]

preferably to give a mixture of compound (III) and compound (V), or a mixture of compound (IV) and compound (V), or a mixture of compound (III), compound (IV), and compound (V),
with the proviso that the reaction mixture formed in step (b) does not comprise any strong acid having a pKₐ value of <4 and not being a carboxylic acid,
(c) distilling off parts or all, preferably all, of the formic acid contained in the mixture obtained in step (b),
(d) washing the distillation residue obtained in step (c) with aqueous sodium hydroxide and/or aqueous potassium hydroxide, preferably until compound(s) (III), (IV), and/or (V) (if present) are at least partially saponified and/or, if applicable, preferably until any traces of formic acid present in the residue obtained in step (c) from previous steps are neutralised, and collecting the organic phase,
(e) washing the organic phase collected in step (d) with aqueous sodium carbonate and/or aqueous sodium hydrogen carbonate, preferably until compound(s) (III), (IV), and/or (V) (if present) are fully saponified and/or preferably until the organic phase is adjusted to a pH of from above 7 to 12, and collecting the organic phase,
(f) fractionally distilling the organic phase collected in step (e) and collecting the purified ethylhexylglycerol of formula (I).

During the studies underlying the present invention and contrary to the teachings of the state of the art, it was surprisingly found that the reaction between 2-ethylhexyl glycidyl ether of formula (II) and formic acid in step (b) of the process according to the invention efficiently takes place in the absence of any strong acids having a pKₐ value of <4 and not being a carboxylic acid (such as e.g. sulfuric acid). This advantageously enables a broader use of existing production reactors in the industrial sector, as no catalyst with extreme corrosive properties is needed.

Step (f) of the process according to the invention delivers the desired clear, colourless and, above all, odourless quality of ethylhexylglycerol of formula (I), which presents a significant advantage over the state of the art.

According to a preferred embodiment, the formic acid provided in step (a) and reacted in step (b) of the process according to the invention—especially in the first reaction cycle of the process according to the invention—is neat formic acid, preferably is formic acid with a purity of over 80%, more preferably of over 90%, more preferably of over 95%, more preferably of over 98%, most preferably of over 99%.

According to a preferred embodiment, the distillation residue obtained in step (c) is washed with aqueous sodium hydroxide and then the organic phase is collected in step (d) of the process according to the invention.

According to another preferred embodiment, the distillation residue obtained in step (c) is washed with aqueous potassium hydroxide and then the organic phase is collected in step (d) of the process according to the invention.

According to another preferred embodiment, the distillation residue obtained in step (c) is washed with a mixture of aqueous sodium hydroxide and aqueous potassium hydroxide and then the organic phase is collected in step (d) of the process according to the invention.

According to a preferred embodiment, in step (d) of the process according to the invention, the distillation residue obtained in step (c) is washed with aqueous sodium hydroxide until compound(s) (III), (IV), and/or (V) (if present) are at least partially saponified, and then the organic phase is collected.

According to a preferred embodiment, in step (d) of the process according to the invention, the distillation residue obtained in step (c) is washed with aqueous sodium hydroxide until compound(s) (III), (IV), and (V) (if present) are at least partially saponified, and then the organic phase is collected.

According to another preferred embodiment, in step (d) of the process according to the invention, the distillation residue obtained in step (c) is washed with aqueous sodium hydroxide until any traces of formic acid present in the residue from previous steps are neutralised, and then the organic phase is collected.

According to a most preferred embodiment, in step (d) of the process according to the invention, the distillation residue obtained in step (c) is washed with aqueous sodium hydroxide until compound(s) (III), (IV), and/or (V) (if present) are at least partially saponified and until any traces of formic acid present in the residue from previous steps are neutralised, and then the organic phase is collected.

According to a most preferred embodiment, in step (d) of the process according to the invention, the distillation residue obtained in step (c) is washed with aqueous sodium hydroxide until compound(s) (III), (IV), and (V) (if present) are at least partially saponified and until any traces of formic acid present in the residue from previous steps are neutralised, and then the organic phase is collected.

According to a preferred embodiment, in step (d) of the process according to the invention, the distillation residue obtained in step (c) is washed with aqueous potassium hydroxide until compound(s) (III), (IV), and/or (V) (if present) are at least partially saponified, and then the organic phase is collected.

According to a preferred embodiment, in step (d) of the process according to the invention, the distillation residue obtained in step (c) is washed with aqueous potassium hydroxide until compound(s) (III), (IV), and (V) (if present) are at least partially saponified, and then the organic phase is collected.

According to another preferred embodiment, in step (d) of the process according to the invention, the distillation residue obtained in step (c) is washed with aqueous potassium hydroxide until any traces of formic acid present in the residue from previous steps are neutralised, and then the organic phase is collected.

According to a most preferred embodiment, in step (d) of the process according to the invention, the distillation residue obtained in step (c) is washed with aqueous potassium hydroxide until compound(s) (III), (IV), and/or (V) (if present) are at least partially saponified and until any traces of formic acid present in the residue from previous steps are neutralised, and then the organic phase is collected.

According to another most preferred embodiment, in step (d) of the process according to the invention, the distillation residue obtained in step (c) is washed with aqueous potassium hydroxide until compound(s) (III), (IV), and (V) (if present) are at least partially saponified and until any traces of formic acid present in the residue from previous steps are neutralised, and then the organic phase is collected.

According to a preferred embodiment, in step (d) of the process according to the invention, the distillation residue obtained in step (c) is washed with a mixture of aqueous sodium hydroxide and aqueous potassium hydroxide until compound(s) (III), (IV), and/or (V) (if present) are at least partially saponified, and then the organic phase is collected.

According to a preferred embodiment, in step (d) of the process according to the invention, the distillation residue obtained in step (c) is washed with a mixture of aqueous sodium hydroxide and aqueous potassium hydroxide until compound(s) (III), (IV), and (V) (if present) are at least partially saponified, and then the organic phase is collected.

According to another preferred embodiment, in step (d) of the process according to the invention, the distillation residue obtained in step (c) is washed with a mixture of aqueous sodium hydroxide and aqueous potassium hydroxide until any traces of formic acid present in the residue from previous steps are neutralised, and then the organic phase is collected.

According to a most preferred embodiment, in step (d) of the process according to the invention, the distillation residue obtained in step (c) is washed with a mixture of aqueous sodium hydroxide and aqueous potassium hydroxide until compound(s) (III), (IV), and/or (V) (if present) are at least partially saponified and until any traces of formic acid present in the residue from previous steps are neutralised, and then the organic phase is collected.

According to another most preferred embodiment, in step (d) of the process according to the invention, the distillation residue obtained in step (c) is washed with a mixture of aqueous sodium hydroxide and aqueous potassium hydroxide until compound(s) (III), (IV), and (V) (if present) are at least partially saponified and until any traces of formic acid present in the residue from previous steps are neutralised, and then the organic phase is collected.

According to a preferred embodiment, only a partial saponification of compound(s) (III), (IV), and/or (V) (if present) takes place in step (d) of the process according to the invention.

According to a further preferred embodiment, only a partial saponification of compound(s) (III), (IV), and (V) (if present) takes place in step (d) of the process according to the invention.

According to a further preferred embodiment, only a partial saponification of compound(s) (III) and (V) takes place in step (d) of the process according to the invention.

According to a further preferred embodiment, only a partial saponification of compound(s) (IV) and (V) takes place in step (d) of the process according to the invention.

According to a preferred embodiment, the organic phase collected in step (d) is washed with aqueous sodium carbonate and then the organic phase is collected in step (e) of the process according to the invention.

According to a preferred embodiment, the organic phase collected in step (d) is washed with aqueous sodium hydrogen carbonate and then the organic phase is collected in step (e) of the process according to the invention.

According to a preferred embodiment, the organic phase collected in step (d) is washed with a mixture of aqueous sodium carbonate and aqueous sodium hydrogen carbonate and then the organic phase is collected in step (e) of the process according to the invention.

According to a preferred embodiment, in step (e) of the process according to the invention, the organic phase collected in step (d) is washed with aqueous sodium hydrogen carbonate until compound(s) (III), (IV), and/or (V) (if present) are (essentially) fully saponified, and then the organic phase is collected.

According to a preferred embodiment, in step (e) of the process according to the invention, the organic phase collected in step (d) is washed with aqueous sodium hydrogen carbonate until compound(s) (III), (IV), and (V) (if present) are (essentially) fully saponified, and then the organic phase is collected.

According to another preferred embodiment, in step (e) of the process according to the invention, the organic phase collected in step (d) is washed with aqueous sodium hydrogen carbonate until the organic phase is adjusted to a pH of from above 7 to 12, preferably from above 7 to 9, most preferably from above 7 to 8, and then the organic phase is collected.

According to a most preferred embodiment, in step (e) of the process according to the invention, the organic phase collected in step (d) is washed with aqueous sodium hydrogen carbonate until compound(s) (III), (IV), and/or (V) (if present) are (essentially) fully saponified and until the organic phase is adjusted to a pH of from above 7 to 12, preferably from above 7 to 9, most preferably from above 7 to 8, and then the organic phase is collected.

According to another most preferred embodiment, in step (e) of the process according to the invention, the organic phase collected in step (d) is washed with aqueous sodium hydrogen carbonate until compound(s) (III), (IV), and (V) (if present) are (essentially) fully saponified and until the organic phase is adjusted to a pH of from above 7 to 12, preferably from above 7 to 9, most preferably from above 7 to 8, and then the organic phase is collected.

According to a preferred embodiment, in step (e) of the process according to the invention, the organic phase collected in step (d) is washed with aqueous sodium carbonate until compound(s) (III), (IV), and/or (V) (if present) are (essentially) fully saponified, and then the organic phase is collected.

According to a preferred embodiment, in step (e) of the process according to the invention, the organic phase collected in step (d) is washed with aqueous sodium carbonate until compound(s) (III), (IV), and (V) (if present) are (essentially) fully saponified, and then the organic phase is collected.

According to another preferred embodiment, in step (e) of the process according to the invention, the organic phase collected in step (d) is washed with aqueous sodium carbonate until the organic phase is adjusted to a pH of from above 7 to 12, preferably from above 7 to 9, most preferably from above 7 to 8, and then the organic phase is collected.

According to a most preferred embodiment, in step (e) of the process according to the invention, the organic phase collected in step (d) is washed with aqueous sodium carbonate until compound(s) (III), (IV), and/or (V) (if present) are (essentially) fully saponified and until the organic phase is adjusted to a pH of from above 7 to 12, preferably from above 7 to 9, most preferably from above 7 to 8, and then the organic phase is collected.

According to a most preferred embodiment, in step (e) of the process according to the invention, the organic phase collected in step (d) is washed with aqueous sodium carbonate until compound(s) (III), (IV), and (V) (if present) are (essentially) fully saponified and until the organic phase is adjusted to a pH of from above 7 to 12, preferably from above 7 to 9, most preferably from above 7 to 8, and then the organic phase is collected.

According to a preferred embodiment, in step (e) of the process according to the invention, the organic phase collected in step (d) is washed with a mixture of aqueous sodium carbonate and aqueous sodium hydrogen carbonate until compound(s) (III), (IV), and/or (V) (if present) are (essentially) fully saponified, and then the organic phase is collected.

According to a preferred embodiment, in step (e) of the process according to the invention, the organic phase collected in step (d) is washed with a mixture of aqueous sodium carbonate and aqueous sodium hydrogen carbonate until compound(s) (III), (IV), and (V) (if present) are (essentially) fully saponified, and then the organic phase is collected.

According to another preferred embodiment, in step (e) of the process according to the invention, the organic phase collected in step (d) is washed with a mixture of aqueous sodium carbonate and aqueous sodium hydrogen carbonate until the organic phase is adjusted to a pH of from above 7 to 12, preferably from above 7 to 9, most preferably from above 7 to 8, and then the organic phase is collected.

According to a most preferred embodiment, in step (e) of the process according to the invention, the organic phase collected in step (d) is washed with a mixture of aqueous sodium carbonate and aqueous sodium hydrogen carbonate until compound(s) (III), (IV), and/or (V) (if present) are (essentially) fully saponified and until the organic phase is adjusted to a pH of from above 7 to 12, preferably from above 7 to 9, most preferably from above 7 to 8, and then the organic phase is collected.

According to a most preferred embodiment, in step (e) of the process according to the invention, the organic phase collected in step (d) is washed with a mixture of aqueous sodium carbonate and aqueous sodium hydrogen carbonate until compound(s) (III), (IV), and (V) (if present) are (essentially) fully saponified and until the organic phase is adjusted to a pH of from above 7 to 12, preferably from above 7 to 9, most preferably from above 7 to 8, and then the organic phase is collected.

According to a preferred embodiment of the process according to the present invention, no alcohol is present in step (d), i.e. no transesterification of compound(s) (III), (IV), and/or (V) (if present) takes place in said step.

According to a preferred embodiment of the process according to the present invention, no alcohol is present in step (d), i.e. no transesterification of compound(s) (III), (IV), and (V) (if present) takes place in said step.

Preferably, no methanol is present in step (d).

Most preferably, no methanol, ethanol, propanol, and butanol are present in step (d).

According to a preferred embodiment of the process according to the present invention, no alcohol is present in step (e), i.e. no transesterification of compound(s) (III), (IV), and/or (V) (if present) takes place in said step.

According to a preferred embodiment of the process according to the present invention, no alcohol is present in step (e), i.e. no transesterification of compound(s) (III), (IV), and (V) (if present) takes place in said step.

Preferably, no methanol is present in step (e).

Most preferably, no methanol, ethanol, propanol, and butanol are present in step (e).

According to a most preferred embodiment of the process according to the present invention, no alcohol is present in steps (d) and (e), i.e. no transesterification of compound(s) (III), (IV), and/or (V) (if present) takes place in said steps.

According to a most preferred embodiment of the process according to the present invention, no alcohol is present in steps (d) and (e), i.e. no transesterification of compound(s) (III), (IV), and (V) (if present) takes place in said steps.

Preferably, no methanol is present in steps (d) and (e).

Most preferably, no methanol, ethanol, propanol, and butanol are present in steps (d) and (e).

Preferably, the reaction mixture of step (b) of the process according to the invention does not comprise any acetates, particularly does not comprise any sodium acetate.

Preferably, the reaction mixture of step (b) also comprises up to 50 wt.-% of water, based on the total weight of the reaction mixture obtained in step (b) (i.e. after complete addition of the 2-ethylhexyl glycidyl ether of formula (II) to the formic acid).

During the studies underlying the present invention, it was surprisingly found that the formic acid, which is separated off in distillation step (c) of the process according to the invention, can be re-used in steps (a) and (b) of a subsequent reaction cycle of the process according to the invention. This leads to a more economical and environmentally friendly synthesis procedure. Due to the elimination of water in the reaction taking place in step (b) of the process according to the invention, the formic acid obtained in distillation step (c) usually has a higher water content than the formic acid provided in step (a) and reacted in step (b) of the process according to the invention. During the studies underlying the present invention, it was surprisingly found that a water content of up to 50 wt.-%—e.g. introduced by recycled formic acid—is tolerated in the reaction mixture of step (b) of the process according to the invention without significantly reducing the yield of ethylhexylglycerol of formula (I) in step (f), despite the state of the art teaching away from the presence of any water in the reaction taking place in step (b) of the process according to the invention.

According to a preferred embodiment, however, the reaction mixture of step (b) comprises less than 0.1 moles of water per mole of 2-ethylhexyl glycidyl ether of formula (II).

According to another embodiment, the reaction mixture of step (b) comprises between 0 and 2 moles, preferably between more than 0 and 2 moles, more preferably between 1 and 2 moles, of water per mole of 2-ethylhexyl glycidyl ether of formula (II).

According to another embodiment of the process according to the invention, the reaction mixture of step (b) comprises less than 50 wt.-%, preferably less than 40 wt. %, more preferably less than 30 wt. %, more preferably less than 20 wt. %, more preferably less than 10 wt. %, more preferably less than 5 wt. %, most preferably less than 1 wt. %, of water, based on the total weight of the reaction mixture obtained in step (b).

According to another embodiment of the process according to the invention, the reaction mixture obtained in step (b) is essentially free of (any externally added) water.

Preferably, the molar ratio of the formic acid to the 2-ethylhexyl glycidyl ether of formula (II) in the reaction mixture obtained in step (b) (i.e. after complete addition of the 2-ethylhexyl glycidyl ether of formula (II) to the formic acid) is between 1:1 and 4:1.

This means that according to a preferred embodiment of the process according to the invention, the molar ratio of the formic acid to the 2-ethylhexyl glycidyl ether of formula (II) provided in step (a) is between 1:1 and 4:1 and the molar ratio of the formic acid to the 2-ethylhexyl glycidyl ether of formula (II) in the reaction mixture obtained in step (b) also is between 1:1 and 4:1.

In step (b) of the process according to the invention, a ring opening reaction of the epoxide functionality of the 2-ethylhexyl glycidyl ether of formula (II) takes place. It is preferred to minimise the dimerisation/trimerisation of the reactants to suppress the formation of by-products. This can be achieved by, preferably slowly (e.g. portionwise or dropwise), adding the 2-ethylhexyl glycidyl ether of formula (II) to the, preferably pre-heated (as defined below), formic acid. Due to sufficient dilution (by formic acid) and a spontaneous reaction, the concentration of 2-ethylhexylglycidyl ether is low at any time, so that the potential for the formation of polymeric by-products decreases.

Preferably, the reaction mixture of step (b) of the process according to the invention does not comprise any organic solvents (other than the formic acid present in the reaction mixture).

The formic acid used in the process according to the present invention simultaneously acts as a (diluting) solvent for the 2-ethylhexyl glycidyl ether of formula (II) and as a reactant for the formation of one or more compound(s) selected from the group consisting of compound (III), compound (IV), and compound (V).

Preferably, the reaction mixture of step (b) of the process according to the invention does not comprise any strong acid selected from the group consisting of H₂SO₄, H₃PO₄, HCl, HNO₃, HClO₄, HBr, HI, and Lewis acids, preferably selected from AlCl₃, BF₃·OEt₂, ZnCl₂.

This is particularly advantageous as it ensures that the corrosion of the material of industrial reactors, that may be used to carry out the process according to the invention, is minimised.

According to a preferred embodiment of step (b) of the process according to the invention, the formic acid is heated to a temperature of from 80 to 110° C., preferably of from 90 to 110° C., more preferably of from 95 to 100° C., before the, preferably dropwise or portionwise, addition of the 2-ethylhexyl glycidyl ether of formula (II).

Preferably, the reaction of step (b) of the process according to the invention, more preferably the addition of the 2-ethylhexyl glycidyl ether of formula (II) to the formic acid in step (b), is carried out over a period of from 10 to 300 min, preferably from 30 to 180 min, most preferably from 60 to 120 min.

As mentioned above, a slow (i.e. dropwise or portionwise) addition of the 2-ethylhexyl glycidyl ether of formula (II) to the, preferably pre-heated (as defined above), formic acid in step (b) of the process according to the invention is particularly advantageous as it reduces the formation of undesired by-products and thus reduces yield losses of ethylhexylglycerol of formula (I). It therefore supports the obtainment of a maximal yield of ethylhexylglycerol of formula (I).

According to a preferred embodiment, the 2-ethylhexyl glycidyl ether of formula (II) reacts with the, preferably pre-heated (as defined above), formic acid immediately upon addition to the formic acid. Thus, according to a preferred embodiment, the time of addition of the 2-ethylhexyl glycidyl ether of formula (II) to the formic acid essentially corresponds to the time of the reaction of step (b). According to an alternative embodiment, the reaction mixture obtained in step (b) may be stirred or rested for some time after the addition of the 2-ethylhexyl glycidyl ether of formula (II) to the formic acid is completed before carrying out step (c) of the method according to the invention.

According to a preferred embodiment of the process according to the invention, the (washing) mixture formed in step (d) comprises a total amount of between 1 and 2 moles, preferably between 1.3 and 1.8 moles, of sodium hydroxide and/or potassium hydroxide per mole of 2-ethylhexyl glycidyl ether of formula (II) as provided in step (a) (and reacted in step (b)).

Preferably, the (washing) mixture formed in step (d) comprises a total amount of between 1 and 2 moles, preferably between 1.3 and 1.8 moles, of sodium hydroxide per mole of 2-ethylhexyl glycidyl ether of formula (II) as provided in step (a) (and reacted in step (b)).

Preferably, the (washing) mixture formed in step (d) comprises a total amount of between 1 and 2 moles, preferably between 1.3 and 1.8 moles, of potassium hydroxide per mole of 2-ethylhexyl glycidyl ether of formula (II) as provided in step (a) (and reacted in step (b)).

More preferably, the (washing) mixture formed in step (d) comprises a total amount of between 1 and 2 moles, preferably between 1.3 and 1.8 moles, of sodium hydroxide and potassium hydroxide per mole of 2-ethylhexyl glycidyl ether of formula (II) as provided in step (a) (and reacted in step (b)).

Washing with the aqueous sodium hydroxide and/or aqueous potassium hydroxide in step (d) of the process according to the invention preferably leads to an only partial saponification of compound(s) (III), (IV), and (V) (if present). The use of an amount of sodium hydroxide and/or potassium hydroxide as defined herein in step (d) of the process is particularly advantageous since the use of a total amount of more than 2 moles of sodium hydroxide and/or potassium hydroxide per mole of 2-ethylhexyl glycidyl ether of formula (II) as provided in step (a) may lead to spontaneous polymerisation of the reactants. Therefore, applying a second washing step with aqueous sodium carbonate and/or aqueous sodium hydrogen carbonate, as defined in step (e) of the process according to the invention, is particularly advantageous as it supports a safe and complete conversion of the reactants into the raw product ethylhexylglycerol of formula (I).

Preferably, the (washing) mixture formed in step (e) comprises a total amount of between 0.1 and 0.5 moles, preferably between 0.2 and 0.4 moles, of sodium carbonate and/or sodium hydrogen carbonate per mole of 2-ethylhexyl glycidyl ether of formula (II) as provided in step (a) (and reacted in step (b)).

Preferably, the (washing) mixture formed in step (e) comprises a total amount of between 0.1 and 0.5 moles, preferably between 0.2 and 0.4 moles, of sodium carbonate per mole of 2-ethylhexyl glycidyl ether of formula (II) as provided in step (a) (and reacted in step (b)).

Preferably, the (washing) mixture formed in step (e) comprises a total amount of between 0.1 and 0.5 moles, preferably between 0.2 and 0.4 moles, of sodium hydrogen carbonate per mole of 2-ethylhexyl glycidyl ether of formula (II) as provided in step (a) (and reacted in step (b)).

Most preferably, the (washing) mixture formed in step (e) comprises a total amount of between 0.1 and 0.5 moles, preferably between 0.2 and 0.4 moles, of sodium carbonate and sodium hydrogen carbonate per mole of 2-ethylhexyl glycidyl ether of formula (II) as provided in step (a) (and reacted in step (b)).

Preferably, the organic phase of step (f), i.e. the organic phase that is fractionally distilled in step (f), is at a pH in the range of from above 7 to 12, preferably of from above 7 to 9, more preferably of from above 7 to 8.

Carrying out the distillation step (f) in the defined pH range is particularly advantageous as it keeps the ethylhexylglycerol of formula (I) in a stable condition.

According to a preferred embodiment of the process according to the invention, washing step (d) is carried out at a temperature of from 20 to 60° C., preferably from 25 to 50° C., more preferably from 30 to 40° C. The defined temperature ranges relate to the temperature of the washing mixture formed during washing step (d). External cooling or heating, preferably cooling, may be required to keep the temperature of the washing mixture in the defined range.

Preferably, washing step (e) is carried out at a temperature of from 20 to 80° C., preferably from 40 to 80° C., more preferably from 50 to 70° C. The defined temperature ranges relate to the temperature of the washing mixture formed during washing step (e). External cooling or heating, preferably heating, may be required to keep the temperature of the washing mixture in the defined range.

According to a preferred embodiment of the process according to the invention, the fractional distillation of step (f) takes place at a pressure of from 0 to 30 mbar, preferably from 2 to 5 mbar, most preferably of about 3 mbar.

Preferably, the fractional distillation of step (f) takes place at a temperature of from 110 to 180° C., preferably from 145 to 160° C., most preferably of about 150° C.

Most preferably, the fractional distillation of step (f) takes place at a pressure of from 0 to 30 mbar, preferably from 2 to 5 mbar, most preferably of about 3 mbar, and at a temperature of from 110 to 180° C., preferably from 145 to 160° C., most preferably of about 150° C.

A second aspect of the invention relates to ethylhexylglycerol of formula (I)

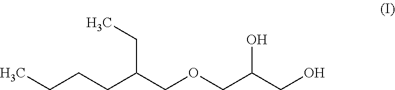

obtained or obtainable according to a process as defined herein.

Advantageously, the process according to the present invention delivers a particularly clear, colourless and, above all, odourless quality of ethylhexylglycerol of formula (I), which presents a significant advantage over the state of the art.

Preferred embodiments of the process according to the invention correspond to or can be derived from the preferred embodiments of the ethylhexylglycerol of formula (I) according to the invention which are explained above or vice versa.

The invention will now be described in more detail hereinafter with references to the examples. Further aspects of the present invention are disclosed in the accompanying claims.

### EXAMPLES

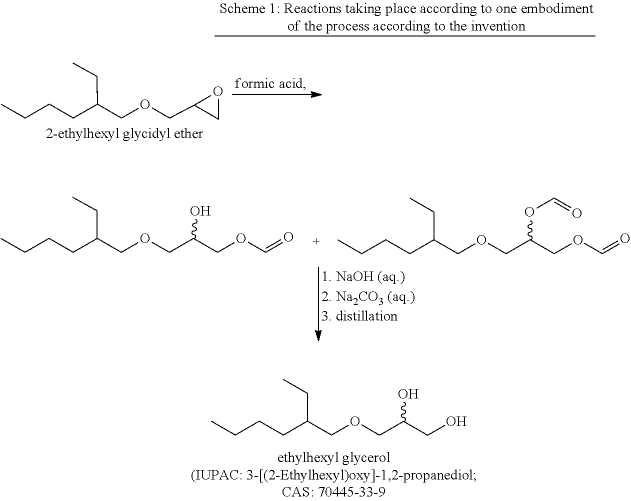 [SMILES: CCCCC(CC)COCC1CO1 ; CCCCC(CC)COCC(O)COC=O ; CCCCC(CC)COCC(COC=O)OC=O ; CCCCC(CC)COCC(O)CO]

### Example 1

1 mol of 2-ethylhexyl glycidyl ether was added over a period of 1 h to 3.8 mol of formic acid at 95-100° C. The exothermic reaction proceeded during the dosing period. The excess of formic acid was distilled of. The crude reaction mixture was saponified in the following washing step with 1.6 mol aqueous sodium hydroxide (30 wt. % in H₂O) and a following treatment with 0.1 mol of sodium carbonate (10 wt. % in H₂O). The slightly basic organic phase (pH 8-9) was distilled (T=130-160° C., p=2-6 mbar) to give clear, colourless, and odourless ethylhexylglycerol with a high purity of 99.8% and an overall yield of 75%.

### Example 2

1 mol of 2-ethylhexyl glycidyl ether was added over a period of 1 h to 3.5 mol of formic acid with 10 wt. % water (from a recycling process) at 95-100° C. The exothermic reaction proceeded during the dosing period. The excess of formic acid was distilled of. The crude reaction mixture was saponified in the following washing step with 1.3 mol aqueous sodium hydroxide (30 wt. % in H₂O) and a following treatment with 0.1 mol of sodium carbonate (10 wt. % in H₂O). The slightly basic organic phase (pH 8-9) was distilled (T=130-160° C., p=2-6 mbar) to give clear, colourless, and odourless ethylhexylglycerol with a high purity of 99.8% and an overall yield of 73%.

### Example 3

1 mol of 2-ethylhexyl glycidyl ether was added over a period of 1 h to 3.8 mol of formic acid with 50 wt. % water at 95-100° C. The exothermic reaction proceeded during the dosing period. The excess of formic acid was distilled of. The crude reaction mixture was saponified in the following washing step with 1.0 mol aqueous sodium hydroxide (30 wt. % in H₂O) and a following treatment with 0.1 mol of sodium carbonate (10 wt. % in H₂O). The slightly basic organic phase (pH 8-9) was distilled (T=130-160° C., p=2-6 mbar) to give clear, colourless, and odourless ethylhexylglycerol with a high purity of 99.8% and an overall yield of 67%.

### Example 4

1 mol of 2-ethylhexyl glycidyl ether was added over a period of 1 h to 3.0 mol of formic acid at 95-100° C. The exothermic reaction proceeded during the dosing period. The excess of formic acid was distilled of. The crude reaction mixture was saponified in the following washing step with 1.4 mol aqueous sodium hydroxide (30 wt. % in H₂O) and a following treatment with 0.1 mol of sodium carbonate (10 wt. % in H₂O). The slightly basic organic phase (pH 8-9) was distilled (T=130-160° C., p=2-6 mbar) to give clear, colourless, and odourless ethylhexylglycerol with a high purity of 99.8% and an overall yield of 70%.

### Example 5

1 mol of 2-ethylhexyl glycidyl ether was added over a period of 4 h to 3.5 mol of formic acid with 10 wt. % water (from a recycling process) at 95-100° C. The exothermic reaction proceeded during the dosing period. The excess of formic acid was distilled of. The crude reaction mixture was saponified in the following washing step with 1.3 mol aqueous sodium hydroxide (30 wt. % in H₂O) and a following treatment with 0.1 mol of sodium carbonate (10 wt. % in H₂O). The slightly basic organic phase (pH 8-9) was distilled (T=130-160° C., p=2-6 mbar) to give clear, colourless, and odourless ethylhexylglycerol with a high purity of 99.5% and an overall yield of 74%.

1-15. (canceled)

16. A process for preparing an ethylhexylglycerol of formula (I) comprising: 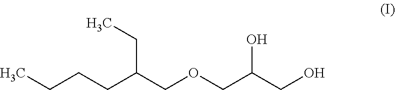
(a) providing 2-ethylhexyl glycidyl ether of formula (II) and formic acid; 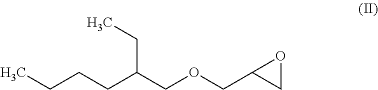
(b) adding the 2-ethylhexyl glycidyl ether of formula (II) to the formic acid, thereby forming a reaction mixture comprising one or more compounds selected from compound (III), compound (IV), and compound (V): 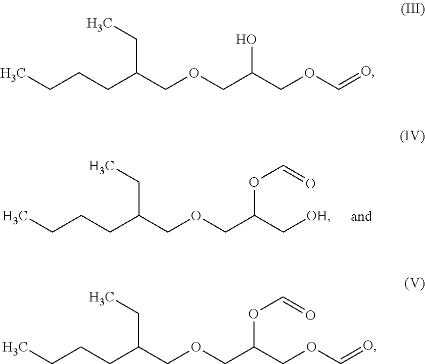 [SMILES: CCCCC(CC)COCC(O)COC=O ; CCCCC(CC)COCC(CO)OC=O ; CCCCC(CC)COCC(COC=O)OC=O]
with the proviso that the reaction mixture does not comprise a strong acid having a pKₐ value of <4 and not being a carboxylic acid;
(c) distilling off at least part of the formic acid from the reaction mixture, thereby obtaining a distillation residue;
(d) washing the distillation residue with aqueous sodium hydroxide and/or aqueous potassium hydroxide and collecting an organic phase;
(e) washing the organic phase with aqueous sodium carbonate and/or aqueous sodium hydrogen carbonate and collecting the organic phase;
(f) fractionally distilling the organic phase from step (e) and collecting purified ethylhexylglycerol of formula (I).

17. The process of claim 16, wherein the reaction mixture of step (b) comprises up to 50 wt. % of water, based on the total weight of the reaction mixture.

18. The process of claim 16, wherein the molar ratio of the formic acid to the 2-ethylhexyl glycidyl ether in the reaction mixture of step (b) is from 1:1 to 4:1.

19. The process of claim 16, wherein the reaction mixture of step (b) does not comprise an organic solvent.

20. The process of claim 16, wherein the reaction mixture of step (b) does not comprise a strong acid selected from sulfuric acid, phosphoric acid, hydrochloric acid, nitric acid, perchloric acid, hydrobromic acid, hydroiodic acid, aluminum chloride, boron trifluoride etherate, and zinc chloride.

21. The process of claim 16, wherein in step (b) the formic acid is heated to a temperature of 80 to 110° C. before the addition of the 2-ethylhexyl glycidyl ether.

22. The process of claim 16, wherein the reaction of step (b) is carried out for 10 to 300 minutes.

23. The process of claim 16, wherein the reaction of step (b) is carried out for 30 to 120 minutes.

24. The process of claim 16, wherein the mixture formed in step (d) comprises a total amount of 1 to 2 moles of sodium hydroxide or potassium hydroxide per mole of 2-ethylhexyl glycidyl ether provided in step (a).

25. The process of claim 16, wherein the mixture formed in step (e) comprises a total amount of 0.1 to 0.5 moles of sodium carbonate or sodium hydrogen carbonate per mole of 2-ethylhexyl glycidyl ether provided in step (a).

26. The process of claim 16, wherein the mixture formed in step (e) comprises a total amount of 0.2 to 0.4 moles of sodium carbonate or sodium hydrogen carbonate per mole of 2-ethylhexyl glycidyl ether provided in step (a).

27. The process of claim 16, wherein the organic phase of step (f) has a pH in the range of greater than 7 to 12.

28. The process of claim 16, wherein washing step (d) is carried out at a temperature of 20 to 60° C.

29. The process of claim 16, wherein washing step (e) is carried out at a temperature of 20 to 80° C.

30. The process of claim 16, wherein washing step (e) is carried out at a temperature of 50 to 70° C.

31. The process of claim 16, wherein the fractional distillation of step (f) takes place at a pressure of 0 to 30 mbar.

32. The process of claim 16, wherein the fractional distillation of step (f) takes place at a pressure of 2 to 5 mbar.

33. The process of claim 16, wherein the fractional distillation of step (f) takes place at a temperature of 110 to 180° C.

34. The process of claim 16, wherein the fractional distillation of step (f) takes place at a temperature of 145 to 160° C.

35. Ethylhexylglycerol of formula (I) obtained or obtainable by the process of claim 16.
